(19) United States
(12) Patent Application Publication
(10) Pub. No.: US 2025/0110110 A1
(43) Pub. Date: Apr. 3, 2025

(54) Point-of-care device for erythrocyte sedimentation monitoring and evaluation of factors that affect erythrocyte sedimentation rate

(71) Applicant: Vitalii Efremov, Novi Sad (RS)

(72) Inventor: Vitalii Efremov, Novi Sad (RS)

(21) Appl. No.: 18/730,703

(22) PCT Filed: Jan. 22, 2023
(86) PCT No.: PCT/RU2023/050005

§ 371 Date: Jul. 19, 2024

(30) Foreign Application Priority Data
Jan. 21, 2022 (RU) 2022101406

(51) Int. Cl. G01N 33/49; G01N 11/00; G01N 11/02

(52) U.S. Cl. CPC G01N 33/49; G01N 11/02; G01N 33/4905; G01N 2011/008

(57) ABSTRACT

Blood analyzing device including a blood container with a fluidic channel for whole blood sample flow, fluidic channel including two sub-channels, the first sub-channel for erythrocyte sedimentation monitoring, hematocrit, fibrinogen and plasma viscosity measurement, the second sub-channel for fibrinogen measurement; a pumping system connected to the channel, to control velocity of the sample flow; two sets of light sources and optical sensors coupled with the channel that measure intensity of light scattered by the sample; signal acquisition electronic module providing amplification of electronic signals acquired from optical sensors and data transfer to a data analyzing processor; data analyzing processor that receives data from signal acquisition electronic module in real-time, stores data in memory, processes data and generates erythrocyte sedimentation time course as an digital array of optical intensity values formed for a certain period of time, erythrocyte sedimentation rate value, hematocrit level value, plasma viscosity value and fibrinogen concentration value.

### TECHNICAL FIELD

The present invention generally relates to blood analysis, and in particular to a blood analyzing device capable of monitoring the process of erythrocyte sedimentation, determining an erythrocyte sedimentation rate with a simultaneous evaluation of three factors affecting erythrocyte sedimentation, namely, hematocrit level, plasma viscosity and fibrinogen concentration in a blood sample.

### BACKGROUND

Blood is commonly described as a complex red fluid consisting of plasma with a suspension of cells, mostly erythrocytes, called hematocrit. A hematocrit level is typically evaluated as a volume fraction taking from 10 to 80% of total blood volume. The hematocrit level is important diagnostic parameter representing capability of blood for oxygen transfer that is of critical importance in blood banking and critical care departments for blood transfusion at trauma, birth delivery etc. Plasma consists mainly of water with salts, proteins, sugars, etc. Even a very small sample of blood taken from one person is a representative amount of blood cells for making measurements to determine certain properties and conditions of the blood. Erythrocyte sedimentation rate (ESR) test is a widely used screening test that enables a non-specific detection of a pathological process ongoing in the organism such as inflammation, chronic diseases of various etiology, among others. Usually test is performed by placing anti-coagulated blood in an upright Westergren tube and the rate at which the red blood cells fall is measured in mm/h. When an inflammatory or other pathological state process is present, the high amount of protein, first of all fibrinogen and C-reactive protein is produced in human organism and exposed into blood. Such proteins have a net charge that is opposite to a charge of erythrocyte membrane, and if blood doesn't move as in in vitro test in tube, erythrocytes stick to each other forming rouleaux stacks which settle faster than individual erythrocytes, thereby resulting in a higher ESR rate.

The conventional Westergren method is cumbersome and slow. It requires at least one hour to completion and wet chemistry space in the lab and some amount of manual operations performed by a user. Thus, this method is not employable in operating theatres and critical care units there it should be performed much faster, 7 minutes as maximum, with a minimal required space and minimal cumbersomeness. The improvements present in [U.S. Pat. No. 6,336,358B1], [CN105547937A], [CN86105076A] and [EP2836820A1] reduce the time required for the ESR test to about 5-15 minutes, improve significantly user-friendliness and enable a reduction of equipment dimensions and, thus, potentially can be used in critical care units as point-of-care rapid and compact devices.

However, these improvements do not solve the main ESR test problem, non-specificity, that limits the use of the test in current practice. The non-specificity and the corresponding lack of interpretability is caused by the fact that ESR is affected not only by the concentration of inflammatory proteins but also by hematocrit level and plasma viscosity which in turn depends on total plasma protein content and multiple other less significant factors. Furthermore, it is impossible to distinguish between the effects of various inflammatory proteins on ESR test result, e.g. fibrinogen vs C-reactive protein. The improvement present in [CN103149125B] discloses a device that provides a simultaneous measurement of ESR with plasma viscosity and hematocrit. This arrangement is based on a blood fractioning performed on a compact centrifuge coupled with optical sensors that measure light absorption by various blood fractions. This solution, however, is not optimal since the test time is still higher than 7 minutes, the compactness and cumbersomeness of a device based on this arrangement are disputed. In addition, it does not provide information about concentration of at least one inflammatory protein affecting the ESR test result. Moreover, mechanical systems as centrifuges are usually less robust than ones based on electronic pumps, less cost effective and more power consuming.

The improvements present in [U.S. Pat. No. 8,900,514B2] is not centrifuge-based, it provides the rapid testing within the 7 minutes time frame, enables compact and robust implementation. Furthermore, it allows for spectroscopic measurement of hemoglobin that is indirectly related to hematocrit level. Hemoglobin concentration though is less informative for erythrocyte sedimentation analysis than hematocrit level since it doesn't relate to mechanical properties of blood and electrical properties of erythrocyte membrane. Moreover this solution doesn't provide information about plasma viscosity and the concentration of at least one inflammatory protein affecting the ESR test result.

### SUMMARY OF THE INVENTION

The above-identified issues are addressed by providing a device for erythrocyte sedimentation monitoring comprising:
a blood container comprising a fluidic channel arranged for whole blood sample flow comprising two parallel sub-channels where the second sub-channel provides a mixing of the blood sample with at least one reagent triggering the contact coagulation pathway such as silica or kaolin, among others;
a pumping system that is connected to the channel and controls velocity of the sample flow supporting a switch between at least two modes of the flow, namely a no-flow mode where the velocity is zero and a steady-flow mode where the velocity has a certain constant value supported by the pumping system during a certain period of time;
two sets of optical sensors coupled with the channel that measure intensity of light scattered by the sample, where the first set comprises at least two sensors located in series along the first sub-channel with certain distance intervals between the consecutive sensors, and the second set comprises one or more sensors located in series along the second sub-channel;
two sets of light sources providing light into the sample aligned with the two sets optical sensors respectively;
a signal acquisition electronic module comprising one or more signal acquisition units providing amplification of electronic signals acquired from the sets of the optical sensors, signal transformation to digital data sets and the data transfer to a data analyzing processor;
a data analyzing processor that receives the data from the signal acquisition electronic module in real-time, stores the data in processor memory, processes the data and generates the results, where the following steps of the data processing are implemented:
the data from the first set of optical sensors acquired during the steady-flow mode is processed in order to identify optical signal shifts detected by each individual sensor as the sample reaches the sensor caused by the difference in optical turbidity between whole blood and any gas or fluid that is present in the sub-channel prior the test, and respective time values where such optical signal shifts occur;
a passing time value as a time interval between the signal shifts detected by any two sensors of the first set of sensors is measured;
a first hematocrit estimator value is defined as an average optical turbidity of the flowing sample and derived from the data acquired from the first set of optical sensors at the steady-flow mode by a comparison of an average signal detected by at least one sensor during a certain period of time to a reference data set of previously measured average signal values detected by the same sensor which data set is obtained for whole blood samples of known hematocrit levels which reference data set is stored in the data analyzing processor memory prior the test;
a blood viscosity value is derived from the passing time value by a comparison of such value to a reference data set of previously measured passing time values obtained for fluidic samples of known viscosities which reference data set is stored in the data analyzing processor memory prior the test;
an erythrocyte sedimentation time course is formed from the real-time data acquired from the first set of sensors during the no flow mode and recorded to the data analyzing processor memory which time course is then fitted by a multi-parametric analytical function which function has at least one parameter identifying an erythrocyte sedimentation rate as a value defining a gradual signal change over time as the erythrocyte sedimentation progresses during a certain period of time until the end of sedimentation when the most of erythrocytes are settled and the optical signal does not significantly change;
the erythrocyte sedimentation rate is derived from the best fit function;
a second hematocrit estimator value is defined as an average optical turbidity of not flowing sample and derived from the best fit function as an end-point signal value corresponding to the end of sedimentation by a comparison of such value to a reference data set of previously measured end-point signal values which data set is obtained for whole blood samples of known hematocrit levels which reference data set is stored in the data analyzing processor memory prior the test;
a hematocrit level is calculated as a linear combination of the first and the second hematocrit estimator values;
a plasma viscosity value is derived from the hematocrit level and the blood viscosity value by a comparison of such set of two values to a reference data set of hematocrit levels and the blood viscosity values previously measured by the same device which data set is obtained for whole blood samples of known plasma viscosity which reference data set is stored in the data analyzing processor memory prior the test;
a clotting time value is defined as a time where optical turbidity of the sample changes significantly in result of fibrin polymerization caused by the sample activation by sample mixing with the reagent triggering the contact coagulation pathway and derived from the signal acquired from at least one of the optical sensors of the second set of sensors during the no-flow mode as a time between the mixing and a corresponding shift of the signal;
a clotting rate value is defined as a parameter indicating how fast the optical turbidity of the sample changes after onset of the fibrin polymerization and derived from the signal acquired from at least one of the optical sensors of the second set of sensors during the no-flow mode as the ratio of the signal change over a certain time interval to the value of such time interval where the first point of such interval corresponds to a time after the clotting time;
a first fibrinogen estimator value is derived from the clotting time value and hematocrit level by comparison of such set of two values to a reference data set of clotting time values and hematocrit levels previously measured by the same device which data set is obtained for whole blood samples of known fibrinogen concentration and hematocrit level which reference data set is stored in the data analyzing processor memory prior the test;
a second fibrinogen estimator value is derived from the clotting rate value and hematocrit level by comparison of such set of two values to a reference data set of clotting rate values and hematocrit levels previously measured by the same device which data set is obtained for whole blood samples of known fibrinogen concentration and hematocrit level which reference data set is stored in the data analyzing processor memory prior the test;
a third fibrinogen estimator value is derived from the erythrocyte sedimentation rate value and hematocrit level by comparison of such set of two values to a reference data set of erythrocyte sedimentation rate values and hematocrit levels previously measured by the same device which data set is obtained for whole blood samples of known fibrinogen concentration and hematocrit level which reference data set is stored in the data analyzing processor memory prior the test;
a fibrinogen concentration is calculated as a linear combination of the first, the second and the third fibrinogen estimator values.

In another aspect, the invention relate to a method for erythrocyte sedimentation monitoring, the method comprising a combination of operations implemented in the device as described above.

In particular aspect, a method according to the inventions is a method for erythrocyte sedimentation monitoring and evaluation of factors that affect erythrocyte sedimentation rate comprising:
obtaining a blood sample from a subject;
placing the sample into a blood container of the device according to claim 1;
actuating the device;
outputting erythrocyte sedimentation rate value;
outputting erythrocyte fibrinogen concentration;
outputting hematocrit level;
outputting plasma viscosity value.

Additional features and advantages of the invention will be set forth in the description that follows, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE ATTACHED FIGURES

The accompanying drawings, which are included to provide a further understanding of the invention and are incorporated in and constitute a part of this specification, illustrate embodiments of the invention and, together with the description, serve to explain the principles of the invention.

In the drawings:

FIG. 1 is a schematic representation of a device for erythrocyte sedimentation monitoring and evaluation of factors that affect erythrocyte sedimentation rate.

FIG. 2 is an operational timeline.

FIG. 3 represents the typical relationships between measured parameters and some characteristics of a whole blood sample.

FIG. 4 represents the steps of fibrinogen concentration estimation based on the erythrocyte sedimentation rate value.

FIG. 5 represents the steps of fibrinogen concentration estimation based on the clotting time value.

FIG. 6 represents the steps of fibrinogen concentration estimation based on the clotting rate value.

FIG. 7 represents the benchmarking of the fibrinogen concentration estimation disclosed herein with a gold standard conventional laboratory method of fibrinogen determination, von Clauss assay.

FIG. 8 is an optional image of how user interface may look like.

### DETAILED DESCRIPTION OF EMBODIMENTS OF THE INVENTION

Reference will now be made in detail to the preferred embodiments of the present invention, examples of which are illustrated in the accompanying drawings.

The present invention overcomes the issues and drawbacks of the prior art arrangements disclosed in [U.S. Pat. No. 8,900,514B2] and [CN103149125B]. The device disclosed herein allows for rapid blood testing, within less 7 minutes timeframe implemented in a compact, cost effective, power consumption effective user friendly format, which blood testing comprises the recording erythrocyte sedimentation time course, determining erythrocyte sedimentation rate value and the list of three values affecting erythrocyte sedimentation such as plasma viscosity, fibrinogen concentration and hematocrit.

Particular embodiments of the above-identified device include the following.

The invention further relates the device as defined above, wherein the second fluidic sub-channel comprises a reservoir with a reagent triggering non-contact coagulation pathway such as thrombin, tissue factor, among others.

In another aspect, the invention relates the device as defined above, wherein the first fluidic sub-channels comprise a series of at least one reservoir with stabilizing reagents such as buffer solution, water, polysaccharide solution, among others, that can improve reproducibility of the test by increasing stability of the sample flow, reproducibility of the sedimentation time course fitting and reproducibility of the clotting time and rate measurement.

In a further aspect, the invention relates the device as defined above, wherein fluidic channel comprises a third, reference sub-channel, with a respective third set of optical sensors, which is identical to the second sub-channel wherein the reservoir of the third sub-channel contains a buffer solution instead of a reagent activating coagulation which buffer solution can be phosphate buffer saline, imidazole among others, or water taken at the same volume as the activating reagent in the second channel, and the clotting time parameter is derived from the signal acquired from the second set of sensors by its comparison to a reference signal acquired from the third set of sensors.

In any of the above aspects, the device may be configured for outputting any combination of erythrocyte sedimentation rate value, erythrocyte fibrinogen concentration, hematocrit level. plasma viscosity value, preferably all of sedimentation rate value, erythrocyte fibrinogen concentration, hematocrit level. plasma viscosity value. The output values will be the ones determined by the device. The claimed invention will be further illustrated with reference to drawings, wherein.

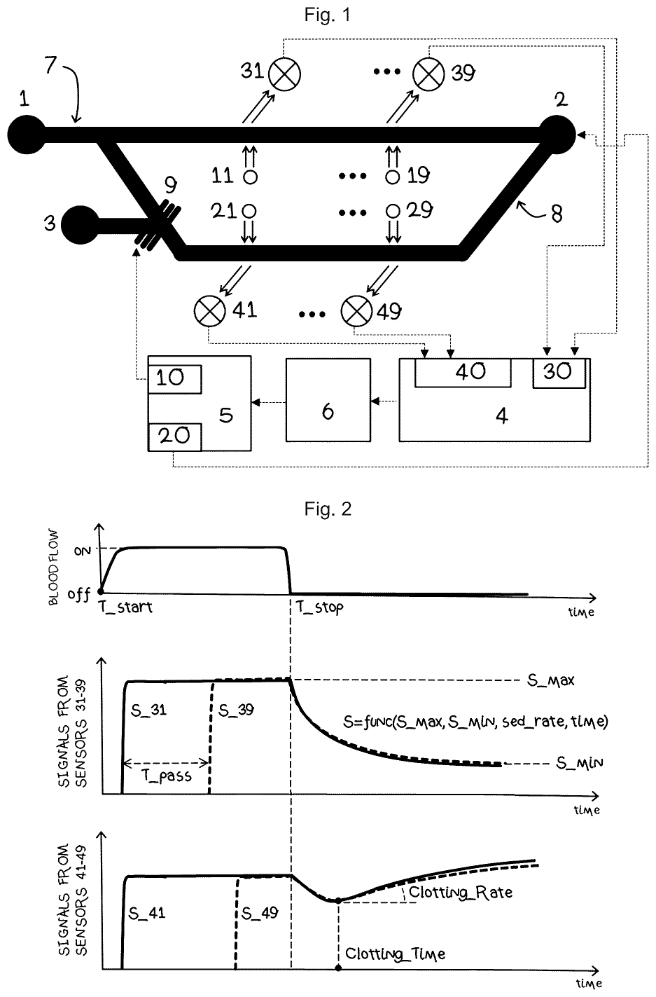

FIG. 1 is a schematic representation of a device for erythrocyte sedimentation monitoring and evaluation of factors that affect erythrocyte sedimentation rate where 1—inlet hole where the blood sample is placed in before the start of a test; 2—outlet hole/waste collection coupled with pump; 3—a reservoir containing coagulation activating reagents such as kaolin or silica; 4—signal acquisition electronic module that acquires electronic signals from optical sensors and transforms them into digital data sets for the following transfer to a data analyzing processor; 5—fluid flow operations control unit that is connected to the pump as well as to valve/mixer; 6—data analyzing processor that receives the data from the signal acquisition electronic module and sends the flow control commands to the pumping system; 7—fluidic channel; 8—second parallel fluidic sub-channel aimed to provide measurements of clotting time and rate after the sample is mixed with the coagulation activation reagent; 9—valve/mixer controlled by the fluid flow operations control unit that provides the effective mixing of the sample with the coagulation activation reagent; 10—electronic sub-unit of the fluid flow operations control unit that sends commands to the valve/mixer; 11-19—light sources that spot the first fluidic sub-channel; 20—electronic sub-unit of the fluid flow operations control unit that sends commands to the pump; 21-29—light sources that spot the second fluidic sub-channel; 30—unit of the signal acquisition electronic module coupled with the first set of sensors; 31-39—first set of optical sensors detecting the light scattered by the sample, coupled with the first fluidic sub-channel and aligned with respective light spots; 40—unit of the signal acquisition electronic module coupled with the second set of sensors; 41-49—second set of optical sensors detecting the light scattered by the sample, coupled with the second fluidic sub-channel and aligned with respective light spots.

Typical total length of the channel is from 1 to 50 cm, preferably from 5 to 40 cm, most preferably from 5 to 30 cm. Typical width of a sub-channel is from 0.1 to 20 mm, preferably from 0.5 to 10 mm, most preferably from 1 to 5 mm. Typical depth of a sub-channel is from 0.02 to 10 mm, preferably from 0.05 to 1 mm, most preferably from 0.1 to 0.5 mm. The pump can be an actively controlled syringe pump, peristaltic pump or other type of a controlled pump or it can be a passive pump like degas driven flow system or a passive capillary system where the switch between no-flow and steady flow modes can be implemented with a number of fluidic valves. The blood container can be implemented in a form of disposable cartridge with the inlet hole for blood sample introduction, which cartridge can have dimensions from 0.1×0.5×2 cm to 5×5×20 cm, preferably to 2×4×10 cm, most preferably to 1×3×5 cm. The coagulation activation reagent, kaolin and/or silica can be taken as a solution in buffer in volume that provides the final concentration in the sample after mixing of from 0.01 to 500 g/L, preferably from 1 to 100 g/L, most preferably from 3 to 30 g/L where the buffer can be an imidazole buffer, phosphate buffer saline, sodium chloride solution in water, among others. The blood sample volume required for the test can be from 0.1 to 1000 uL, preferably from 1 to 100 uL, most preferably from 10 to 50 uL.

FIG. 2 is an operational timeline. The pump operates at two modes, namely, “no-flow” where velocity of the sample flow is zero and “steady-flow” where velocity is kept at a constant value higher than zero, which modes are indicated here as “off” and “on” respectively. The fluidic channel is empty before the start of the test, time<T_start, and the sensors of the first set of optical sensors detect no signal here. As soon as blood riches the spot of the first sensor it detects a massive light scattering by the blood and the corresponding signal shifts up to S_max value. The same shifts are detected by all other sensors of the first set of sensors with some delays, which delays depend on velocity of blood flow that in turn directly depends on blood viscosity. The delay between any two sensors, T_pass, can be measured and then analyzed by a processor in order to derive the blood viscosity value. Sensors of the second set behave similarly where the magnitude of the shift and delay times may differ since the blood sample riches the sensor spots after some dilution in result of mixing with the coagulation activation reagent, and hence, such sample has lower density and optical turbidity and higher viscosity. The signal obtained from any sensor of the first set starts to decay dramatically as soon as the fluid flow stops, i.e. where pump turns into the no-flow mode. A time course of the signal observed at T>T_stop represents erythrocyte sedimentation kinetics. The time course can be fitted by some analytical function in order to extract parameters of a rate of such decay, sed_rate, and an end-point of sedimentation, S_min. The signal obtained from any sensor of the second set also starts decaying for some time until the clot formation onsets. Such clotting is initiated by the coagulation activation reagent and typically delayed in action by its nature, from 10 to 500 seconds from the time of mixing depending on type and concentration of the reagent and on hemostatic properties of the sample. Such clotting increases optical turbidity that increases the corresponding signal value. The time where such increase becomes significant is defined as a clotting time and the rate of increase is defined as a clotting rate.

The data analyzing processor that receives the data from the signal acquisition electronic module in real-time, stores the data in processor memory, processes the data and generates the results, where the following steps of the data processing are implemented:
the data from the first set of optical sensors acquired during the steady-flow mode is processed in order to identify optical signal shifts detected by each individual sensor as the sample reaches the sensor caused by the difference in optical turbidity between whole blood and any gas or fluid that is present in the sub-channel prior the test, and respective time values where such optical signal shifts occur;
a passing time value, T_pass, as a time interval between the signal shifts detected by any two sensors of the first set of sensors is measured;
a first hematocrit estimator value is defined as an average optical turbidity of the flowing sample and derived from the data acquired from the first set of optical sensors at the steady-flow mode by a comparison of an average signal detected by at least one sensor, S_max, during a certain period of time to a reference data set of previously measured average signal values detected by the same sensor which data set is obtained for whole blood samples of known hematocrit levels which reference data set is stored in the data analyzing processor memory prior the test;
a blood viscosity value is derived from the passing time value by a comparison of such value to a reference data set of previously measured passing time values obtained for fluidic samples of known viscosities which reference data set is stored in the data analyzing processor memory prior the test;
an erythrocyte sedimentation time course is formed from the real-time data acquired from the first set of sensors during the no flow mode and recorded to the data analyzing processor memory which time course is then fitted by a multi-parametric analytical function which function has at least one parameter identifying an erythrocyte sedimentation rate as a value defining a gradual signal change over time as the erythrocyte sedimentation progresses during a certain period of time until the end of sedimentation when the most of erythrocytes are settled and the optical signal does not significantly change;
the erythrocyte sedimentation rate is derived from the best fit function;
a second hematocrit estimator value is defined as an average optical turbidity of not flowing sample, S_min, and derived from the best fit function as an end-point signal value corresponding to the end of sedimentation by a comparison of such value to a reference data set of previously measured end-point signal values which data set is obtained for whole blood samples of known hematocrit levels which reference data set is stored in the data analyzing processor memory prior the test;
a hematocrit level is calculated as a linear combination of the first and the second hematocrit estimator values;
a plasma viscosity value is derived from the hematocrit level and the blood viscosity value by a comparison of such set of two values to a reference data set of hematocrit levels and the blood viscosity values previously measured by the same device which data set is obtained for whole blood samples of known plasma viscosity which reference data set is stored in the data analyzing processor memory prior the test;
a clotting time value is defined as a time where optical turbidity of the sample changes significantly in result of fibrin polymerization caused by the sample activation by sample mixing with the reagent triggering the contact coagulation pathway and derived from the signal acquired from at least one of the optical sensors of the second set of sensors during the no-flow mode as a time between the mixing and a corresponding shift of the signal;
a clotting rate value is defined as a parameter indicating how fast the optical turbidity of the sample changes after onset of the fibrin polymerization and derived from the signal acquired from at least one of the optical sensors of the second set of sensors during the no-flow mode as the ratio of the signal change over a certain time interval to the value of such time interval where the first point of such interval corresponds to a time after the clotting time;
a first fibrinogen estimator value is derived from the clotting time value and hematocrit level by comparison of such set of two values to a reference data set of clotting time values and hematocrit levels previously measured by the same device which data set is obtained for whole blood samples of known fibrinogen concentration and hematocrit level which reference data set is stored in the data analyzing processor memory prior the test;
a second fibrinogen estimator value is derived from the clotting rate value and hematocrit level by comparison of such set of two values to a reference data set of clotting rate values and hematocrit levels previously measured by the same device which data set is obtained for whole blood samples of known fibrinogen concentration and hematocrit level which reference data set is stored in the data analyzing processor memory prior the test;
a third fibrinogen estimator value is derived from the erythrocyte sedimentation rate value and hematocrit level by comparison of such set of two values to a reference data set of erythrocyte sedimentation rate values and hematocrit levels previously measured by the same device which data set is obtained for whole blood samples of known fibrinogen concentration and hematocrit level which reference data set is stored in the data analyzing processor memory prior the test;
a fibrinogen concentration is calculated as a linear combination of the first, the second and the third fibrinogen estimator values.

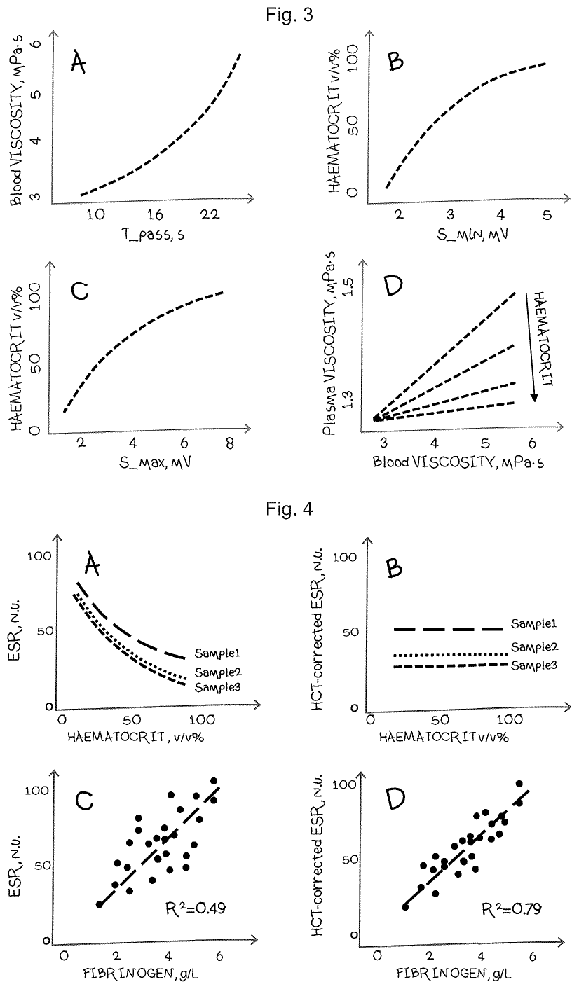

FIG. 3 represents the typical relationships between measured parameters and some characteristics of a whole blood sample. The blood viscosity value can be derived from the T_pass value since there is the strong relationship between two (A). Typical determined T_pass values are from 1 to 200 s, preferably from 2 to 100 s, most preferably from 3 to 30 s. Hematocrit level can be derived from S_max or S_min values or from a combination of two (B, C). Typical determined S_min values are from 0 to 10000 mV, preferably from 0 to 100 mV, most preferably from 0 to 50 mV. A plasma viscosity value can be determined analytically after the blood viscosity and the hematocrit level are determined since there is a direct relationship between plasma viscosity and blood viscosity at every known hematocrit level (D).

FIG. 4 represents the steps of fibrinogen concentration estimation based on the erythrocyte sedimentation rate value. First step is an elimination of the erythrocyte sedimentation rate dependence on hematocrit level (A) to make fibrinogen concentration determination from erythrocyte sedimentation rate more specific. The elimination is performed with analytical function which function is based on analysis of multiple blood samples where each individual blood sample is processed in order to vary its hematocrit level. In brief, such sample processing starts from sample separation to plasma and hematocrit by a standard centrifugation followed by re-suspension of these two substances taken at various proportions to obtain a series of new samples with a range of hematocrit levels where the properties of plasma such as its protein content remain the same. The hematocrit elimination analytical function coverts the dependence of erythrocyte sedimentation rate on hematocrit into a straight line (B). Such function is then becomes a part of a processor software code used for data analysis. In general, erythrocyte sedimentation rate strongly correlates with fibrinogen concentration (C) and the use of hematocrit corrected erythrocyte sedimentation rate makes such correlation much stronger (D). This allows fibrinogen concentration estimation at some level of confidence that can be expressed in terms of coefficient of determination, R2 value, that is generally from 0 to 1, where the higher value means higher specificity and confidence of the estimation. In the case of the device disclosed herein such value can be as high as from 0.6 to 1.0, preferably from 0.7 to 1.0, most preferably from 0.8 to 1.0.

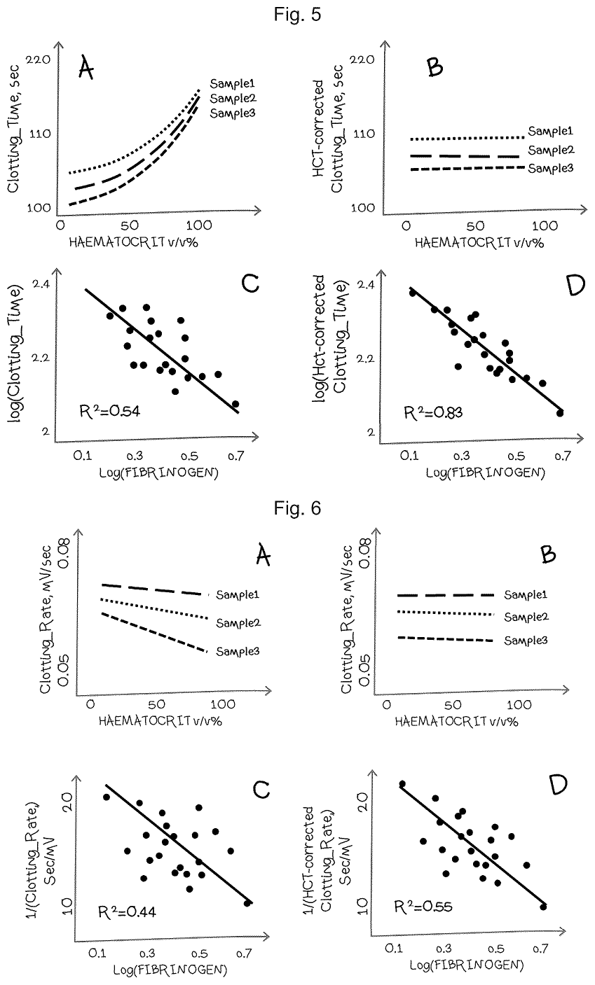

FIG. 5 represents the steps of fibrinogen concentration estimation based on the clotting time value. First step is an elimination of the clotting time value dependence on hematocrit level (A) to make fibrinogen concentration determination more specific. The elimination is performed with analytical function which function is based on analysis of multiple blood samples where each individual blood sample is processed in order to vary its hematocrit level. The hematocrit elimination analytical function coverts the dependence of clotting time value on hematocrit into a straight line (B). Such function is then becomes a part of a processor software code used for data analysis. In general, clotting time value strongly correlates with fibrinogen concentration (C) and the use of hematocrit corrected clotting time value makes such correlation stronger (D). This allows fibrinogen concentration estimation at some level of confidence that can be expressed in terms of coefficient of determination, R2 value, that is generally from 0 to 1, where the higher value means higher specificity and confidence of the estimation. In the case of the device disclosed herein such value can be as high as from 0.6 to 1.0, preferably from 0.7 to 1.0, most preferably from 0.8 to 1.0.

FIG. 6 represents the steps of fibrinogen concentration estimation based on the clotting rate value. First step is an elimination of the clotting rate value dependence on hematocrit level (A) to make fibrinogen concentration determination more specific. The elimination is performed with analytical function which function is based on analysis of multiple blood samples where each individual blood sample is processed in order to vary its hematocrit level. The hematocrit elimination analytical function coverts the dependence of clotting rate value on hematocrit into a straight line (B). Such function is then becomes a part of a processor software code used for data analysis. In general, clotting time rate value correlates with fibrinogen concentration to some extent (C) and the use of hematocrit corrected clotting rate value makes such correlation stronger (D). This allows fibrinogen concentration estimation at some level of confidence that can be expressed in terms of coefficient of determination, R2 value, that is generally from 0 to 1, where the higher value means higher specificity and confidence of the estimation. In the case of the device disclosed herein such value can be as high as from 0.4 to 1.0, preferably from 0.5 to 1.0, most preferably from 0.6 to 1.0.

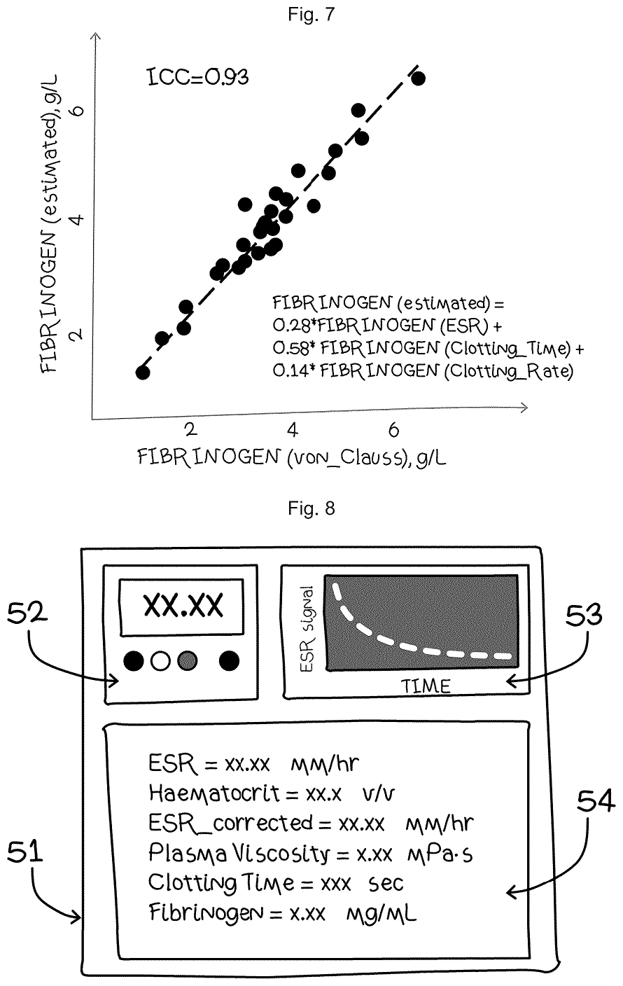

FIG. 7 represents the benchmarking of the fibrinogen concentration estimation disclosed herein with a gold standard conventional laboratory method of fibrinogen determination, von Clauss assay. The linear combination of three fibrinogen estimators, namely derived from erythrocyte sedimentation rate, derived from clotting time and derived from clotting rate, is used here for deriving the final fibrinogen estimator that is more specific and precise when compared to each individual estimator taken separately. An Intraclass Correlation Coefficient for the final estimator versus von Clauss assay values is as high as from 0.8 to 1.0, that can be interpreted as a high level of agreement between two methods.

FIG. 8 is an optional image of how user interface may look like. Here, 51 is the interface panel, 52 is a control and indication board for user interactions with the device, 53—a screen where user can observe and monitor a progress of erythrocyte sedimentation which plot is based on the time course formed by the data analyzing processor in real-time, 54—a screen presenting results to user, first of all, the erythrocyte sedimentation rate expressed in normalized units or aligned with Westergren scale, in mm/hr, that is familiar to most of potential users or in mV/sec or similar units based on signal to time ratio. In addition a series of factors affecting the sedimentation rate are shown to user, namely the hematocrit level value expressed in percentage of volume fraction units; plasma viscosity value expressed in mPas or similar units; clotting time value in seconds or minutes; fibrinogen concentration value in g/L or mg/mL or like.

Having thus described a preferred embodiment, it should be apparent to those skilled in the art that certain advantages of the described method and apparatus have been achieved.

It should also be appreciated that various modifications, adaptations and alternative embodiments thereof may be made within the scope and spirit of the present invention. The invention is further defined by the following claims.

### REFERENCES (ALL INCORPORATED HEREIN BY REFERENCE IN THEIR ENTIRETY)

[U.S. Pat. No. 6,336,358B1] Method and apparatus for measuring sedimentation rate of sediments in liquid sample
[CN105547937A] Automatic control device for detecting erythrocyte sedimentation rate data
[CN86105076A] Blood sampling and measurement erythrocyte sedimentation rate device
[U.S. Pat. No. 8,900,514B2] Device for determining the erythrocyte sedimentation rate in a blood sample
[EP2836820A1] Apparatus, method, system for the determination of the aggregation rate of red blood cells
[CN103149125B] Whole blood and plasma viscosity and blood sedimentation and hematocrit one-machine determination method and equipment

What is claimed is:

1. A device for erythrocyte sedimentation monitoring comprising:
a blood container comprising a fluidic channel arranged for whole blood sample flow comprising two parallel sub-channels where the second sub-channel provides a mixing of the blood sample with at least one reagent triggering the contact coagulation pathway such as silica or kaolin, among others;
a pumping system that is connected to the channel and controls velocity of the sample flow supporting a switch between at least two modes of the flow, namely a no-flow mode where the velocity is zero and a steady-flow mode where the velocity has a certain constant value supported by the pumping system during a certain period of time;
two sets of optical sensors coupled with the channel that measure intensity of light scattered by the sample, where the first set comprises at least two sensors located in series along the first sub-channel with certain distance intervals between the consecutive sensors, and the second set comprises one or more sensors located in series along the second sub-channel;
two sets of light sources providing light into the sample aligned with the two sets optical sensors respectively;
a signal acquisition electronic module comprising one or more signal acquisition units providing amplification of electronic signals acquired from the sets of the optical sensors, signal transformation to digital data sets and the data transfer to a data analyzing processor;
a data analyzing processor that receives the data from the signal acquisition electronic module in real-time, stores the data in processor memory, processes the data and generates the results, where the following steps of the data processing are implemented:
the data from the first set of optical sensors acquired during the steady-flow mode is processed in order to identify optical signal shifts detected by each individual sensor as the sample reaches the sensor caused by the difference in optical turbidity between whole blood and any gas or fluid that is present in the sub-channel prior the test, and respective time values where such optical signal shifts occur;
a passing time value as a time interval between the signal shifts detected by any two sensors of the first set of sensors is measured;
a first hematocrit estimator value is defined as an average optical turbidity of the flowing sample and derived from the data acquired from the first set of optical sensors at the steady-flow mode by a comparison of an average signal detected by at least one sensor during a certain period of time to a reference data set of previously measured average signal values detected by the same sensor which data set is obtained for whole blood samples of known hematocrit levels which reference data set is stored in the data analyzing processor memory prior the test;
a blood viscosity value is derived from the passing time value by a comparison of such value to a reference data set of previously measured passing time values obtained for fluidic samples of known viscosities which reference data set is stored in the data analyzing processor memory prior the test;
an erythrocyte sedimentation time course is formed from the real-time data acquired from the first set of sensors during the no flow mode and recorded to the data analyzing processor memory which time course is then fitted by a multi-parametric analytical function which function has at least one parameter identifying an erythrocyte sedimentation rate as a value defining a gradual signal change over time as the erythrocyte sedimentation progresses during a certain period of time until the end of sedimentation when the most of erythrocytes are settled and the optical signal does not significantly change;
the erythrocyte sedimentation rate is derived from the best fit function;
a second hematocrit estimator value is defined as an average optical turbidity of not flowing sample and derived from the best fit function as an end-point signal value corresponding to the end of sedimentation by a comparison of such value to a reference data set of previously measured end-point signal values which data set is obtained for whole blood samples of known hematocrit levels which reference data set is stored in the data analyzing processor memory prior the test;
a hematocrit level is calculated as a linear combination of the first and the second hematocrit estimator values;
a plasma viscosity value is derived from the hematocrit level and the blood viscosity value by a comparison of such set of two values to a reference data set of hematocrit levels and the blood viscosity values previously measured by the same device which data set is obtained for whole blood samples of known plasma viscosity which reference data set is stored in the data analyzing processor memory prior the test;
a clotting time value is defined as a time where optical turbidity of the sample changes significantly in result of fibrin polymerization caused by the sample activation by sample mixing with the reagent triggering the contact coagulation pathway and derived from the signal acquired from at least one of the optical sensors of the second set of sensors during the no-flow mode as a time between the mixing and a corresponding shift of the signal;
a clotting rate value is defined as a parameter indicating how fast the optical turbidity of the sample changes after onset of the fibrin polymerization and derived from the signal acquired from at least one of the optical sensors of the second set of sensors during the no-flow mode as the ratio of the signal change over a certain time interval to the value of such time interval where the first point of such interval corresponds to a time after the clotting time;
a first fibrinogen estimator value is derived from the clotting time value and hematocrit level by comparison of such set of two values to a reference data set of clotting time values and hematocrit levels previously measured by the same device which data set is obtained for whole blood samples of known fibrinogen concentration and hematocrit level which reference data set is stored in the data analyzing processor memory prior the test;
a second fibrinogen estimator value is derived from the clotting rate value and hematocrit level by comparison of such set of two values to a reference data set of clotting rate values and hematocrit levels previously measured by the same device which data set is obtained for whole blood samples of known fibrinogen concentration and hematocrit level which reference data set is stored in the data analyzing processor memory prior the test;
a third fibrinogen estimator value is derived from the erythrocyte sedimentation rate value and hematocrit level by comparison of such set of two values to a reference data set of erythrocyte sedimentation rate values and hematocrit levels previously measured by the same device which data set is obtained for whole blood samples of known fibrinogen concentration and hematocrit level which reference data set is stored in the data analyzing processor memory prior the test;
a fibrinogen concentration is calculated as a linear combination of the first, the second and the third fibrinogen estimator values.

2. The device according to claim 1, wherein the second fluidic sub-channel comprises a reservoir with a reagent triggering non-contact coagulation pathway such as thrombin, tissue factor, among others.

3. The device according to claim 1, wherein the first fluidic sub-channels comprise a series of at least one reservoir with stabilizing reagents such as buffer solution, water, polysaccharide solution, among others, that can improve reproducibility of the test by increasing stability of the sample flow, reproducibility of the sedimentation time course fitting and reproducibility of the clotting time and rate measurement.

4. The device according to claim 1, wherein fluidic channel comprises a third, reference sub-channel, with a respective third set of optical sensors, which is identical to the second sub-channel wherein the reservoir of the third sub-channel contains a buffer solution instead of a reagent activating coagulation which buffer solution can be phosphate buffer saline, imidazole among others, or water taken at the same volume as the activating reagent in the second channel, and the clotting time parameter is derived from the signal acquired from the second set of sensors by its comparison to a reference signal acquired from the third set of sensors.
